(19) United States
(12) Patent Application Publication
(10) Pub. No.: US 2011/0130738 A1
(43) Pub. Date: Jun. 2, 2011

(54) Absorptive Pad for Use on Wounds

(75) Inventor: Andreas Schmidt, Duderstadt (DE)

(73) Assignee: MCAIRLAID'S VLIESSTOFFE GMBH & CO. KG, Berlingerode (DE)

(21) Appl. No.: 12/954,926

(22) Filed: Nov. 29, 2010

(30) Foreign Application Priority Data
Nov. 30, 2009 (DE) 20 2009 015 670.2

(51) Int. Cl. A61F 13/56; A61L 15/60; A61L 15/58

(52) U.S. Cl. 604/385.03; 604/372

(57) ABSTRACT

An absorptive pad for placement on wounds in the form of a layered body of a multi-layer configuration has a first layer facing the skin, a second layer facing away from the skin, and an absorptive core arranged between the first layer and the second layer. The first layer has a wound area to be placed onto a wound, wherein the wound area of the first layer is made of a material that is permeable to liquids. The first layer outside of the wound area is provided at least partially with an adhesive.

### BACKGROUND OF THE INVENTION

The invention concerns an absorptive pad for placement on wounds, configured as a layered body of a multi-layer configuration, comprising at least a layer A facing the skin, a layer B facing away from the skin, and an absorptive core C arranged between the layer A and the layer B.

Absorptive pads for absorbing bodily liquids are not only used in hygiene but also in medical applications, in particular for the treatment of wounds.

Absorptive pads that are used as wound pads are generally placed onto an exterior wound in order to prevent penetration of foreign matter into the wound and to absorb blood and wound secretions.

US 2005/0143697 A1 discloses an absorptive pad for placement on wounds in particular for absorbing wound secretions. The absorptive pad has a flat material section of absorptive material, i.e., an absorptive nonwoven with superabsorber particles distributed therein as well as a liquid-permeable envelope. The envelope encloses the material section of absorptive material and forms a barrier with respect to solid bodily excretions and prevents passage of other excreted substances to the absorptive material arranged within the envelope. The material section of absorptive material has a surface area that is smaller than the surface area of the envelope, i.e., in the non-wetted state 3% to 75% of the envelope when laid flat. The surface area of the laid-flat envelope is moreover delimited by a seam and optionally by a folded edge. The envelope and/or the absorptive material may be provided on its circumference with a substance that is adhering to the body of the patient such as an adhesive. Pectin-cellulose compounds are disclosed as a suitable adhesive.

The wound pads disclosed in the prior art have the disadvantage that they must be secured on the wound by means of appropriate bandaging material. Also, the wound pads easily become displaced when a bandaging material is applied and when wearing the bandaging material. There is no possibility for securing the wound pad directly to the skin of the patient.

Therefore, the invention has the object to provide an absorptive pad for placement on wounds that has full absorptive capacity but can be secured without further auxiliary means such as bandaging material on the wound or the skin of the patient.

### SUMMARY OF THE INVENTION

The object of the present invention is an absorptive pad of the aforementioned kind that is characterized in that the layer A that is facing the skin in the wound area has a material that is permeable to the liquids and, outside of the wound area, is provided at least partially with an adhesive means.

The absorptive pad according to the invention is suitable in particular for placement on wounds, i.e. onto so-called external wounds, that may usually occur on the skin including mucous membrane of the patient. These wounds may be acute injuries but also chronic wounds, i.e., wounds that are not healing, or wounds that only heal by secondary wound healing.

The absorptive pad according to the invention has the advantage that the wound is covered by a layer that absorbs the excreted bodily liquid wherein this layer in an area outside of the wound area is provided at least partially with an adhesive as an adhesive means. In this way, it is possible to secure the absorptive pad on the body surface without having to employ additional bandaging material etc. The fixation of the absorptive pad without bandaging material has moreover the advantage that the absorptive pad in a simple way can be placed onto the wound and caused to adhere by the patient himself. Should the absorptive pad be additionally secured or covered by a bandaging material or the like, a displacement is prevented during bandaging and also while wearing the pad.

The absorptive pad is a layered body of a multi-layer configuration that is substantially comprised of a layer A facing the skin, a layer B facing away from the skin, and an absorptive body that is positioned between these two layers.

The layer A that is facing the skin is resting immediately on the wound and partially on the skin surface surrounding the wound. In order to ensure an optimal wound care, the layer A is comprised of a liquid-permeable material in the area in which it is immediately resting on the wound. This liquid-permeable material can be selected from materials such as nonwoven or fabric and/or can be a perforated two-dimensional or three-dimensional film, which materials preferably do not stick to the wound. Nonwoven materials are textile-like materials that are produced of long fibers and that are joined with one another by chemical, mechanical, heat or solvent treatment. They are textile web materials of individual fibers that are liquid permeable. In addition to nonwoven materials (fleece), also perforated or structured two-dimensional or three-dimensional films can be used as layer A. The perforated films in their two-dimensional configuration have holes in their surface area that ensure liquid passage through the material. Perforated three-dimensional films are characterized in that by means of the hole structure the perforated material extends across the surface of the web material so that a three-dimensional structure results. As a result of the three-dimensional structure the contact surface on the wound is reduced which has a positive effect on wound healing and prevents sticking together of dried excretions and wound patch. The materials of which the nonwoven/fleece or the perforated films are produced are polyolefin films, such as polyethylene or polypropylene films but also nonwovens of natural materials.

It is important that the layer A in the area outside of the wound/the absorptive core at least partially is provided with an adhesive means and preferably an adhesive. The area of the wound and preferably also the area extending immediately about the wound should however be free of adhesive. In one possible configuration the layer A can be comprised of the already described nonwoven and/or a film that, at its edges, is provided, e.g. coated, with an adhesive that is permanently adhesive.

In a further embodiment the layer A can also be comprised of several zones or of different materials in different zones, wherein only zones outside of the wound/the absorptive core resting on the healthy skin surface are provided with adhesive.

As an adhesive all conceivable skin-compatible adhesives are considered as they are conventional and known for manufacturing wound patches. Preferably, the adhesive should be one that is permanently adhesive so that even after a first use, i.e. when it has already been applied once to the skin surface, it can be removed again and reused. In this way it is possible to readjust the position of the pad again. Also, the adhesive should preferably not lose its adhesive power when coming into contact with water. A preferably employed adhesive is so-called underindexed polyurethane, i.e., a polyurethane with low isocyanate index. Underindexed polyurethanes are obtained by reaction of polyisocyanates with long-chain polyols that are preferably free of any short-chain components.

The topside of the absorptive pad according to the invention, i.e., the side that is facing away from the skin, is formed by the layer B. The latter has preferably the same dimensions as the layer A. In order to be able to join layers A and B with one another, they can be, depending on the material from which these layers are formed, adhesively connected, fused, sealed or connected in other ways.

In a further configuration, the dimensions of one of the two layers A or B are greater than those of the other layer and the projecting rim can be folded over the rim of the other layer in such a way that the external rims can thereby be closed off.

An optimal wound treatment is possible in particular when the layer that is facing away from the skin is also a layer of breathable material.

The materials of which the layers A and B are produced may be the same or different. Preferably, the layer B is breathable, i.e., it is permeable to water vapor but not for liquids. The layer B can be, for example, a breathable film such as a perforated two-dimensional and/or three-dimensional film, a breathable SMS (spunbond/meltblown/spunbond) material and/or a laminate of different materials, for example, of nonwoven and breathable film (BTBS films=breathable film textile backsheet) as they are used conventionally for producing exterior sides of incontinence products.

As a third layer, the absorptive pad according to the invention has an absorptive core. It is enclosed by the layers A and B preferably like an envelope. The absorptive core serves to absorb the excreted exudates and to absorb them permanently. A re-wetting in the direction of the wound should preferably be prevented as much as possible. The absorptive core is therefore formed preferably of a material that is capable not only of absorbing bodily liquids but also of storing them, such as pulp or pulp-related materials as well as synthetic absorptive materials. The absorptive core is preferably a nonwoven of pulp fibers, such as an air-laid product.

In order to increase the absorptive capacity of the absorptive core further, the latter may contain particles of superabsorber polymers. These can be, for example, incorporated directly during manufacture of the absorptive core.

In one embodiment, the absorptive core is comprised of a fiber web of pulp fibers or a loose agglomeration of pulp fibers (fluff pulp) that are laid in an airstream, which are passed through a calander comprised of two embossing rollers. The fibers are calandered in a spot or linear pattern without a binding agent by generating an embossment pattern in the pressure area and are thus connected to one another. An absorptive core that is produced according to the method disclosed in Canadian patent 2 309 998 has been found to be especially suitable.

When the absorptive body C is enclosed by the layers A and B like an envelope, the absorptive body can be secured to one or both of the layers A and B or it can be surrounded loosely by the layers A and B. In order to prevent as much as possible sliding of the absorptive body within this envelope and to ensure that the absorptive body covers at least completely or partially the layer A at the location where it is resting on the wound, the absorptive body should have at least the same size as the wound. In a preferred embodiment, the absorptive body is somewhat smaller than the layer A.

The layers A and B are preferably closed at their outer edges. In one possible embodiment of the present invention. the layers A and B are identical or almost identical and the edges are glued, fused, or sealed together. This connection of the two layers can be realized immediately at the edges of the layers or at a certain spacing from the edges so that essentially a circumferentially extending seam is formed. It is also possible to provide an adhesive connection that extends up to the absorptive core.

In a further embodiment of the invention the surface area of one of the two layers A and B is greater than the other surface area, the projecting surface areas of the larger layer can be folded over the edges of the smaller layer so that an envelope is also formed that encloses the absorptive body.

### BRIEF DESCRIPTION OF THE DRAWINGS

The invention will be explained with the aid of the attached figures in more detail.

FIG. 1 is a view onto an absorptive pad of a flat configuration.

FIG. 2 is an embodiment of an absorptive pad in a section illustration.

FIG. 3 is another embodiment in section illustration with absorptive core engaged between the edge areas of the skin-facing layer and the cover layer.

FIG. 4 is another embodiment in section illustration with an additional layer between absorptive core and cover layer.

FIG. 5 is another embodiment in section illustration with folded over edges of the skin-facing layer.

FIG. 6 is another embodiment in section illustration with folded over edges of the cover layer.

FIG. 7 is another embodiment in section illustration with an additional layer between absorptive core and skin-facing layer.

### DESCRIPTION OF PREFERRED EMBODIMENTS

FIG. 1 shows a view of an absorptive pad 1 viewed in a direction onto the side that is facing the skin when in use. This first side of the absorptive pad will be referred to in the following as layer A.

The first layer 2 that is facing the skin has applied thereto in its outer areas 3, i.e., outside of the wound area W, an adhesive 4. The adhesive 4 should be disposed or applied only in those areas that, in contrast to the centrally arranged area of the layer 2 covering the wound, do not come into contact with the wound itself. In order to not impair the absorptive capacity of the absorptive pad according to the invention, the surfaces of the layer 2 facing the skin should be as much as possible free of coatings so that the permeation of liquids through this layer remains intact. Moreover, there is the risk that, when an adhesive reaches the vicinity of the wound, the healing process may be impaired.

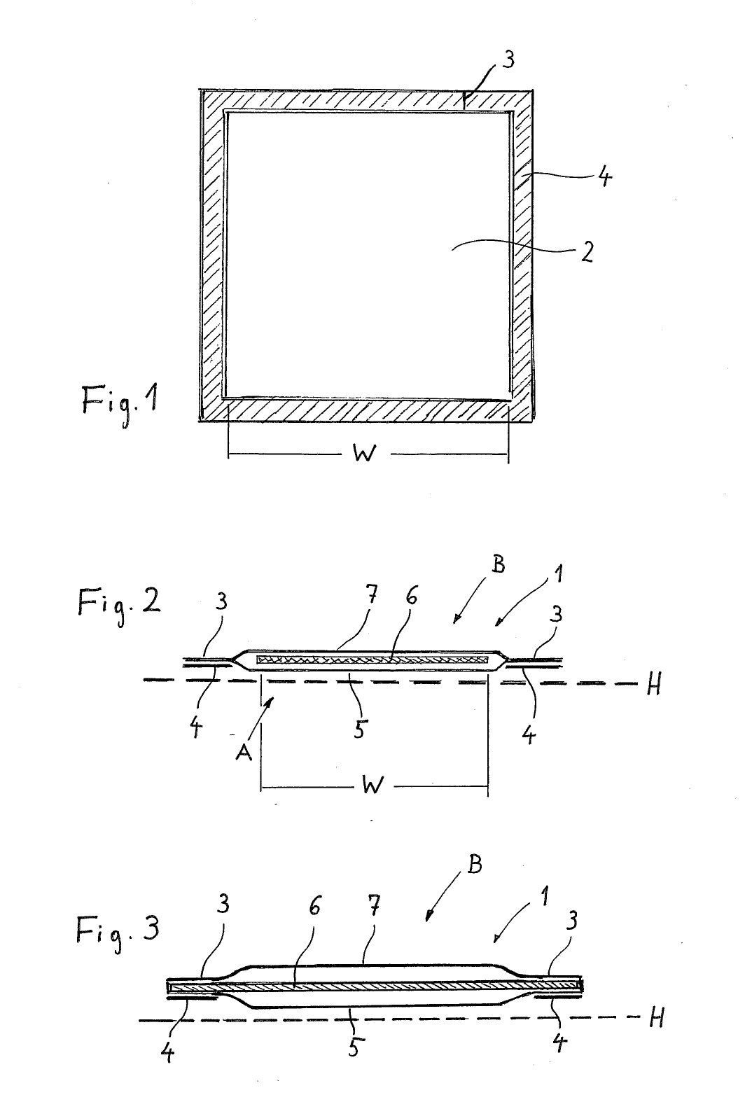

FIG. 2 shows a first embodiment of the absorptive pad in a schematic section view. The absorptive pad is a layered body 1 of a multi-layer configuration with a layer 2 facing the skin H that has an adhesive 4 applied along its outer areas 3. Between the areas 3 to which the adhesive 4 is applied the wound contact surface 5 is located. The wound contact surface 5 is permeable to liquids in order to allow passage of exudates excreted by the wound. The exudates are absorbed by the central absorptive body 6 and stored therein.

On the side that is facing away from the skin H, referred to in the following also as layer B, the absorptive pad has a layer 7 that constitutes the externally positioned cover layer of the absorptive pad. The cover layer 7 is comprised preferably of breathable materials, i.e., materials that are permeable to vapor but are impermeable to liquids. By means of this cover layer that is facing away from the skin it is to be prevented as much as possible that exudate may exit from the absorptive pad and cause soiling e.g. of clothing of the patient.

In the embodiment illustrated in FIG. 2 the layer 2 facing the skin H and the layer 7 facing away from the skin are connected to one another along a rim area 3 that forms a circumferentially extending zone in the illustrated embodiment. The connection of the layers in this zone may be spot-shaped so that the layers 2 and 7 in the external areas are not connected to one another. They can also be connected to each other across the entire surface area. In the area 5 the absorptive pad is resting on the skin H; this area extends across the entire wound and preferably also across the area of the skin that is immediately neighboring the wound area, and optionally past it. In this way, it can be prevented that the wound will come into contact with the adhesive means 4.

In the embodiment illustrated in FIG. 3, the absorptive body extends into close proximity of the outer edges 3 where, when connecting the layers or plies 2 and 7, it is also engaged and in this way is secured between these layers.

In FIG. 4, a further embodiment of the absorptive pad is illustrated. In this embodiment between the absorptive core 6 and the layer 7 that is facing away from the skin H an additional layer or ply 9 is arranged. This layer can be comprised of fine nonwoven or tissue. It is to prevent that liquid can penetrate to the exterior when the absorptive pad has been resting already for a long time on the wound and the absorption capacity is exhausted.

In the embodiment illustrated in FIG. 4, the layers 2 and 7 are connected with each other by partial fusing or by spot fusing. In this way, local connections 10 between the layers 2 and 7 are formed without a further circumferentially extending edge being formed.

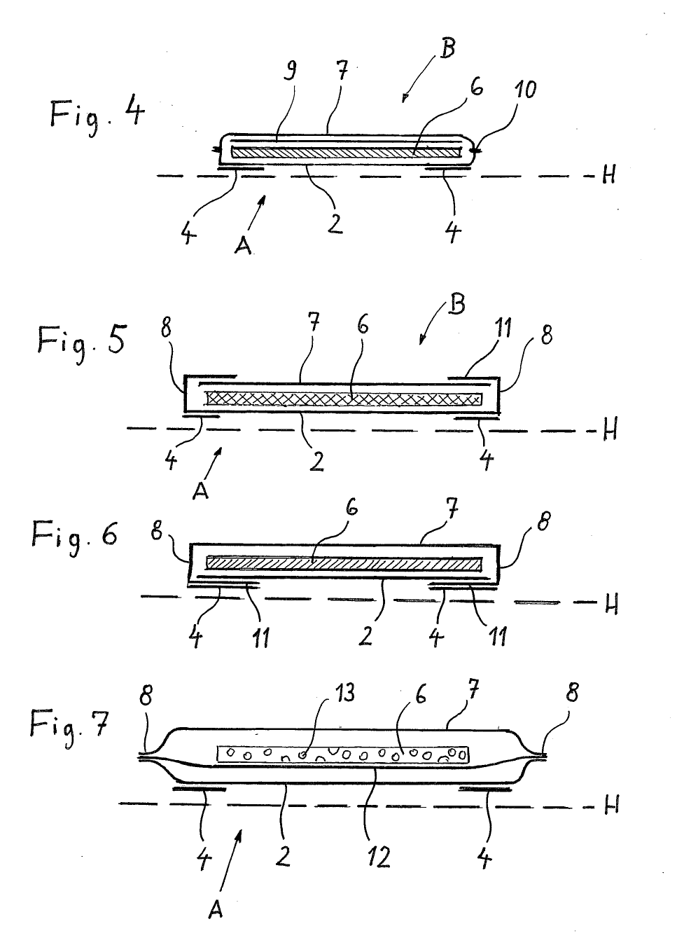

In FIGS. 5 and 6 embodiments are illustrated in which the layers 2 and 7 have different sizes. In the embodiment of FIG. 5, the surface area of the layer that is facing the skin H is greater than that of the layer that is facing away from the skin so that the projecting portions 11 may be folded over and secured to the layer 7 so that closed outer edges 8 are formed.

FIG. 6 shows an embodiment in which the layer 7 that is facing away from the skin H is greater than the layer 2 facing the skin. Therefore, the layer 8 can be folded over and form a closed rim about the layer 2 and about the absorptive body 6.

FIG. 7 shows a further embodiment of the absorptive pad according to the invention. In this embodiment, between the absorptive body 6 and the layer 2 facing the skin H an additional layer 12 is arranged. The latter is preferably a thin nonwoven or tissue that is liquid-permeable to the bodily liquids and exudates to be absorbed. The layer 12 can have, on one hand, the task to prevent that superabsorber particles 13 arranged in the absorptive body 6 can escape in the direction toward the wound. On the other hand, the layer 12 imparts to the absorptive body 6 a certain stability in case that the absorption capacity of the absorptive body 6 is exhausted and, upon further absorption of liquid, there would be the risk that the absorptive core will swell too much and possibly break apart.

The specification incorporates by reference the entire disclosure of German priority document 20 2009 015 670.2 having a filing date of Nov. 30, 2009.

While specific embodiments of the invention have been shown and described in detail to illustrate the inventive principles, it will be understood that the invention may be embodied otherwise without departing from such principles.

What is claimed is:

1. An absorptive pad for placement on wounds, configured as a layered body of a multi-layer configuration, comprising:
a first layer facing the skin;
a second layer facing away from the skin;
an absorptive core arranged between the first layer and the second layer;
wherein the first layer has a wound area to be placed onto a wound, wherein the wound area of the first layer is comprised of a material that is permeable to liquids; and
wherein the first layer outside of the wound area is provided at least partially with an adhesive means.

2. The absorptive pad according to claim 1, wherein the material permeable to liquids is selected from the group consisting of nonwovens, fabrics, perforated two-dimensional films, perforated three-dimensional films, and combinations thereof.

3. The absorptive pad according to claim 1, wherein the adhesive means is an adhesive that is permanently adhesive.

4. The absorptive pad according to claim 3, wherein the adhesive contains underindexed polyurethane.

5. The absorptive pad according to claim 1, wherein the second layer is comprised of a breathable material.

6. The absorptive pad according to claim 5, wherein the breathable material is selected from the group consisting of perforated films, breathable SMS films, nonwovens of natural or synthetic fibers, laminates of nonwoven and breathable films, BTBS films, and combinations thereof.

7. The absorptive pad according to claim 1, wherein the absorptive core is a liquid-storing pulp or a liquid-storing pulp-related material.

8. The absorptive pad according to claim 7, wherein the absorptive core is produced of a fiber web of pulp fibers passed through a calander comprised of two embossing rollers.

9. The absorptive pad according to claim 7, wherein the absorptive core is produced of a loose agglomeration of pulp fibers (fluff pulp) that is produced by laying in an airstream, wherein the loose agglomeration is passed through a calander comprised of two embossing rollers.

10. The absorptive pad according to claim 7, wherein the absorptive core contains particles of superabsorber polymers.

11. The absorptive pad according to claim 1, wherein the first and second layers envelope the absorptive core and wherein the rims of the first and second layers are closed.

12. The absorptive pad according to claim 1, wherein the absorptive core is a web material with dimensions smaller than dimensions of a surface area of the first and second layers, respectively.
